(19) United States
(12) Plant Patent Application Publication
(10) Pub. No.: US 2017/0127597 P1
(43) Pub. Date: May 4, 2017

(54) Campanula plant named 'PKM04'

(50) ;

(71) Applicant: Gartneriet PKM A/S, Odense N (DK)

(72) Inventor: CHRISTIAN HALD MADSEN, Korsor (DK)

(73) Assignee: Gartneriet PKM A/S, Odense N, (DK)

(21) Appl. No.: 14/756,950

(22) Filed: Nov. 2, 2015

(51) Int. Cl. A01H 5/02

(52) U.S. Cl. PLT/414

(57) ABSTRACT

A distinct cultivar of Campanula plant named ‘PKM04’ characterized by its upright, freely branching and compact growth habit and bicolored (white/violet) broad campanulate flowers.

Latin name of genus and species of the plant claimed: Campanula hybrid.

Variety denomination: ‘PKM04’.

### BACKGROUND OF THE INVENTION

The present invention relates to a new and distinct cultivar of Campanula plant, botanically known as Campanula hybrid, common name Bellflower and hereinafter referred to by the name ‘PKM04’.

The new Campanula originated from a planned cross between two different Campanula species; the female parent being an unnamed, proprietary Campanula variety (unpatented) and the male parent being an unnamed, proprietary Campanula variety (unpatented). The initial crossing was performed in 2012 by the Inventor, followed by further testing and selection in 2013 after vegetative propagation.

Vegetative propagation was performed by terminal cuttings taken and propagated in Søhus, Denmark. Asexual reproduction of the new Campanula cultivar by terminal cuttings has demonstrated that the combination of characteristics as herein disclosed for the new cultivar are firmly fixed and retained through successive generations of asexual reproduction. The new cultivar asexually reproduces true-to-type.

### SUMMARY OF THE INVENTION

The following traits have been repeatedly observed and are determined to be the unique characteristics of ‘PKM04’. These characteristics in combination distinguish ‘PKM04’ as a new and distinct cultivar:
1. Upright and freely branching growth habit;
2. Bicolored flowers.
3. Compact growth with no need for growth regulators

Plants of the cultivar ‘PKM04’ can be compared to plants of the Campanula ‘PKM03’ (U.S. Plant patent application Ser. No. 14/544,525). In side-by-side comparisons conducted by the Inventor in Søhus, Denmark, ‘PKM04’ and ‘PKM03’ differ in the following characteristics:
1. ‘PKM04’ produce smaller plants than ‘PKM03’.
2. ‘PKM04’ has darker purple flowers than ‘PKM03’.
3. ‘PKM04’ has darker green leaves than ‘PKM03’
4. ‘PKM04’ has much less pollen than ‘PKM03’, thus the purple styles are visible whereas the styles of ‘PKM03’ are covered with yellowish pollen.

Data on plants of the parent varieties is no longer available for comparison with plants of the new cultivar Campanula ‘PKM04’.

### BRIEF DESCRIPTION OF THE PHOTOGRAPHS

The accompanying colored photographs illustrate the overall appearance of the new Campanula ‘PKM04’, showing the colors as true as it is reasonably possible to obtain in colored reproductions of this type. Colors in the photographs may differ slightly from the color values cited in the detailed botanical description, which more accurately describe the actual colors of ‘PKM04’.

FIG. 1: The photograph comprises a side perspective view of a typical flowering plant of ‘PKM04’, as grown in a 10.5 cm pot.

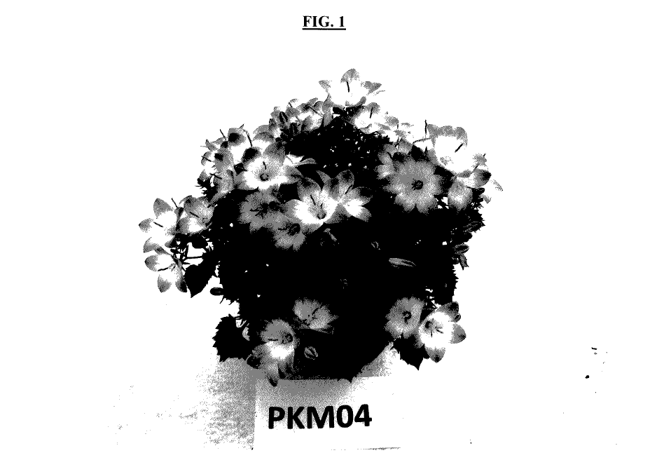

FIG. 2: The photograph comprises a close-up view of a typical inflorescence of ‘PKM04’.

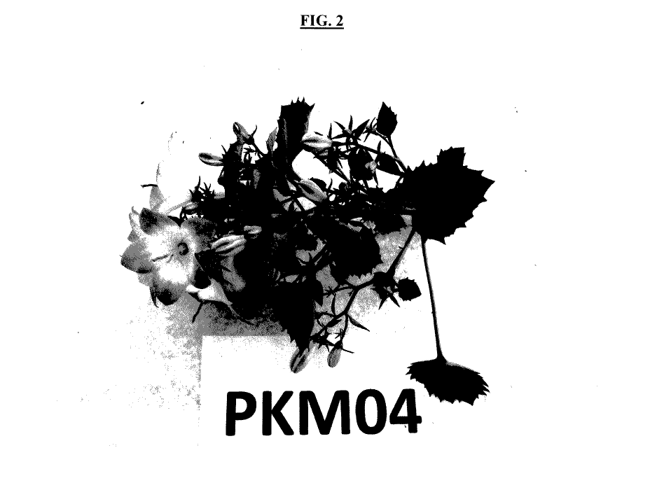

FIG. 3: The photograph comprises a side perspective view of typical flowering plants of ‘PKM04’ and ‘PKM03’, as grown in a 10.5 cm pot.

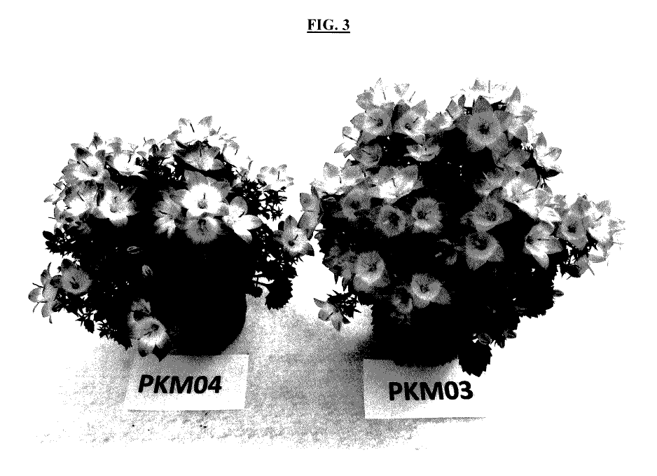

### DETAILED BOTANICAL DESCRIPTION

The new Campanula ‘PKM04’ has not been observed under all possible environmental conditions. The phenotype of the new cultivar may vary with variations in environment such as temperature, light intensity, day length, growth regulator treatment and fertility level without any variance in genotype.

The aforementioned photographs, together with the following observations, measurements and values describe plants of the new Campanula ‘PKM04’ as grown under greenhouse conditions in a 10.5 cm pot.

The age of the ‘PKM04’ plants described is about 14 weeks after propagation. Color references are made to The Royal Horticultural Society Colour Chart (R.H.S.), 5^{th} edition, except where general colors of ordinary significance are used.
Classification:
   Botanical.—Campanula hybrid.
Parentage:
   Female or seed parent.—Unnamed proprietary variety of Campanula (unpatented).
   Male or pollen parent.—Unnamed proprietary variety of Campanula (unpatented).
Propagation:
   Type cutting.—Terminal vegetative cuttings.
   Time to initiate roots.—About 10 to 14 days at 18 to 21° C. in tunnels in a greenhouse.
   Root description.—Fine, well branched.
Plant description:
   Form.—Upright growth habit and overall globular shape. Freely branching with basal shoots. No pinching required.
   Crop time.—After rooting, about 14 weeks are required to produce finished flowering plants in up to 11 cm pots.
   Plant height (from soil level to top of plant plane).—up to 15 cm.
   Plant spread (width).—Up to 24 cm.
   Vigor.—Good. Average growth rate.
   Lateral branches.—Number per plant: 10-15 Length: up to 14 cm (including flowers) Diameter: ca 3 mm Internode length: 5-15 mm.
   Stem.—Shape: Round-edged Strength: Stiff Color: Green, as leaf blade Pubescence: None Lactate: a limited quantity of white lactate is produced.
Foliage description:
   Quantity per lateral branch.—5-10.
   Leaf arrangement.—Single, alternate.
   Leaf shape.—Basal: Cordate Apical: Elliptic to ovate.
   Leaf tip.—Acute (Apical and Basal leaves).
   Leaf base.—Basal: Cordate Apical: Acute to Obtuse.
   Leaf length.—Basal: up to 30 mm Apical: 10-15 mm.
   Leaf width.—Basal: up to 30 mm Apical: 10 mm.
   Pubescence.—: None.
   Margin.—Dentate to Serrate, more prominent on basal than apical leaves.
   Color.—Upper Surface, RHS N137A, Green. Lower Surface, RHS 138B, Green.
   Venation.—Pattern: Pinnipalmate Color: Upper Surface: same as leaf blade, green Lower Surface: same as blade.
   Petiole.—Length: Basal: up to 50 mm Apical: 0-10 mm Diameter: 1-2 mm Color: same as leaf blade, green.
Inflorescence description:
   Flower arrangement and shape.—Broad campanulate flowers. Upright to spreading arrangement. Inflorescences are panicles.
   Flower longevity.—Longevity of individual flowers is highly dependent on temperature and light conditions; average about 2-3 weeks. Flowers persistent. Natural flowering season spring to early summer.
   Inflorescence size.—Length: up to 10 cm.
   Flowers.—Quantity per inflorescence: 12-16 Length: about 20 mm Diameter: 30-40 mm Arrangement and Shape: Single, sympetalous, campanulate. Upright to outward facing. Petal Color: Inner surface: Base: NN155B , White Apex: RHS 88B, Violet. Outer surface: Base: RHS NN155B Apex: RHS 91B. Little darker/more intense at the apex than at the base Color does not fade. Petal number: 5, fused at lower half Petal Shape: Obtuse lobes Petal Margin: Entire Petal Apex: Acute Lobe Length: up to 13 mm lobes Lobe Width: 10-13 mm maximum on lobes Petal texture: Smooth Fragrance: None.
   Sepals.—Arrangement: Free. Appearance: Glabrous Quantity per flower: 5 Length: 5-8 mm. Width: 2 mm. Overall shape: Deltoid. Apex: Acute. Margin: Entire. Color (mature and immature): Upper and lower surfaces: Green, as leaf blade. Pedicel: Length: 5-10 mm Diameter: About 1 mm Color: Green, as leaf blade.
   Buds.—Length: Up to 15 mm Diameter: Up to 7 mm Shape: Oblong, ridged Color: Changes from yellow-green to nearly 88B at the apex. Coloration of buds starts at the tip.
Reproductive organs:
   Pistil.—Quantity: 1 Length: 15-20 mm.
   Style.—Length: 12-18 mm Color: almost white at base (lower half), darker purple than petal upper part in the upper half.
   Ovary.—Color: Green, RHS N137A.
   Stigma.—Shape: Tripartite Color: Greyish Stamen: Number: 5 Anther: Size: 5 mm Color: yellow to light brown Pollen: Amount: None to very little.
High/low temperature tolerance: Plant is not frost tolerant.
Disease resistance and/or susceptibility: Not tested.

I claim:

1. A new and distinct cultivar of Campanula hybrid plant named ‘PKM04’, as illustrated and described herein.
